(19) United States
(12) Patent Application Publication
(10) Pub. No.: US 2023/0077980 A1
(43) Pub. Date: Mar. 16, 2023

(54) ANTI-OBSTRUCTIVE DENTAL ORTHOTIC PRODUCING INCREASED INTRAORAL VOLUME

(71) Applicant: James Metz, Columbus, OH (US)

(72) Inventor: James Metz, Columbus, OH (US)

(21) Appl. No.: 17/472,649

(22) Filed: Sep. 12, 2021

(51) Int. Cl. A61F 5/56

(52) U.S. Cl. CPC A61F 5/566

(57) ABSTRACT

An anti-obstructive airway dental orthotic producing increased intraoral volume. A maxillary retainer is connected to a mandibular retainer by a strut having a length variable only between a fixed minimum and a fixed maximum length. The maxillary retainer has two pivot points such that the strut may be moved from the second pivot point to the first pivot point (to effect a progressive adjustment of mandibular advance. The maxillary retainer may be provided with a spacing area that creates a minimum vertical separation between a cusp of the user's maxillary premolar and a cusp of the user's opposing mandibular premolar. The spacing area provides additional space over the prior art for a lateral expansion of the tongue, which increase intraoral airway volume and eases obstructive sleep apnea. Methods for the attenuation of obstructive sleep apnea (OSA) and for decreasing perioperative morbidity and mortality among OSA patients are described.

### CROSS-REFERENCE TO RELATED APPLICATIONS

The continuation-in-part patent application claims priority to U.S. Utility patent application Ser. No. 15/821,282, filed Nov. 22, 2017, and incorporates that application as if fully rewritten herein.

### STATEMENT REGARDING FEDERALLY SPONSORED RESEARCH OR DEVELOPMENT

Not applicable.

### TECHNICAL FIELD

The present disclosure relates generally to the field of dental orthotics, including a means for the advancement of the lower jaw, relative to the upper, and a means of achieving a minimum vertical separation of the maxillary and mandibular teeth, and in particular, to orthotics producing increased intraoral airway volumes.

### BACKGROUND OF THE INVENTION

A large number of persons have varying degrees of upper airway obstruction present with varied degrees of symptoms. As the upper airway begins with the nose and mouth, it is not surprising that the nose, tongue, and jaw all make contributions to such obstructive syndromes. In particular, it is recently appreciated that the tongue often plays a pivotal role in airway patency.

Various treatment modalities have been suggested. Various airway pressure devices, such as continuous positive airway pressure devices (CPAP) have been successfully used, although these tend to be both cumbersome and uncomfortable. More recently, dental orthotics have been employed. One general class of orthotic relies upon shifting the position of the lower jaw, generally moving the jaw forward, to displace the tongue anteriorly and thereby help clear the upper airway. Obviously, it is difficult to shift the jaw anteriorly without an external anchor point, so these orthotics have also presented problems of utility and comfort.

A classical means for advancement of the jaw is the Herbst appliance. The Herbst appliance is a fixed, tooth-borne, functional orthodontic appliance in which jaw position is influenced by a pin-and-tube spring-loaded appliance that is cemented or bonded to the teeth.

In particular, the Herbst appliance, or Herbst-type orthotics, often suffer from a number of practical problems. Generally, these devices are semi-permanently bonded to the teeth, and while they can be removed by a practitioner, they are not generally amenable to removal by the wearer. These appliances generally utilize spring-loaded struts, in order that a continuous pressure may be applied to advance the jaw.

Other appliances have adjustable displacements formed by either having an adjustable length strut, or by having an adjustable anchor point at one end of a strut, typically a slidable adjustment point, most often seen at the maxillary end of the strut. These devices depend on a practitioner making multiple adjustments, over time, to the degree of displacement of the jaw. The adjustable nature of these devices results in struts or adjustment points that are inherently complex and relatively weak.

### SUMMARY OF THE INVENTION

In its most general configuration, the presently disclosed dental orthotic advances the state of the art with a variety of new capabilities and overcomes many of the shortcomings of prior devices and methods in new and novel ways. In its most general sense, the presently disclosed dental orthotic also overcomes the shortcomings and limitations of the prior art in any of a number of generally effective configurations.

The assessment of a final desired jaw advancement may be made by a number of methods, which may include but are not limited to; subjective sense of airway improvement by the wearer, a measurement of a resting heart rate at an ambient atmosphere of the wearer, the achievement of an advancement to a predetermined amount of advancement by objective metrics, measuring and comparing resting arterial blood oxygen saturation level at an ambient atmosphere of the wearer to predetermined levels, measuring and comparing a resting heart rate at an ambient atmosphere of the wearer to predetermined levels, and perhaps most importantly, an assessment of obstructive sleep apnea events.

Obstructive sleep apnea (OSA) is a prevalent and relatively underdiagnosed condition, which affects 12 to 22% of adults, and is characterized by recurrent episodes of partial and complete airway obstruction during sleep. As of 2012, 82% of men and 93% of women with OSA were estimated to be undiagnosed. The apnea-hypopnea index (AHI), as derived from the overnight polysomnogram, is the most commonly used measure of disease severity, with an AHI<5/hour being considered normal, 5-14.9/hour mild, 15-29.9/hour moderate, and ≥30 events/hour being defined as severe OSA. Patients with OSA are subjected to intermittent hypoxia, sympathetic activation, and sleep fragmentation, which if left untreated are independently associated with cardiometabolic disturbances, neurocognitive deficits, motor vehicle and work-related accidents, insomnia, anxiety and depression, sexual dysfunction, and ultimately, an increased risk for premature death.

Although polysomnography (PSG) is generally considered the “gold standard” diagnostic method, home sleep apnea testing (HSAT) via portable, unattended cardiorespiratory monitors may provide accurate diagnosis and is increasingly utilized. HSAT offers the benefit of cost-efficient diagnosis in the home sleep environment, as well as being recognized as not inferior to PSG.

Dental professionals can be an important part of the multidisciplinary sleep medicine team. The dental office can frequently provide an entry point into the healthcare system for those patients who are not regularly evaluated by a physician, and systematic OSA screening of dental patients can identify those who may warrant referral for suspected OSA. Clinical practice guidelines currently recommend using mandibular advancement devices (MAD) for adults with OSA who are intolerant of continuous positive airway pressure (CPAP) therapy, and dentists with sufficient training can readily provide such therapeutic intervention.

Historically, MADs have been employed to treat snoring and mild to moderate OSA. More recent evidence points to MADs being a viable option even for those patients with severe OSA, especially in cases of poor CPAP compliance. MADs have typically been shown to be less successful in the presence of concurrent obesity. A complete response (CR) to MAD therapy is defined as a post-treatment respiratory event index (REI) of <5 events/hour; existing literature demonstrates that typical CR rates hover around 35-40%.

However, as the studies behind this specification and the inventions resulting from it have shown, thinking exclusively in terms of MAD as a treatment for OSA may be missing part of the picture. In its simplest reduction, the oropharynx may be thought of as a box, whose principal space-occupying structure is the tongue. As has been informally said, the tongue is either in the mouth, or in the throat. Therefore, any intervention, including but not necessarily limited to MAD, that increases the volume of the box, will tend to decrease the chances for OSA. This will be shown to be an important consideration in the specification to follow, particularly but not exclusively, with reference to the concepts of minimizing orthotic size and achieving vertical jaw separation, neither of which is strictly related to mandibular advancement.

Numerous variations, modifications, alternatives, and alterations of the various preferred embodiments, processes, and methods may be used alone or in combination with one another as will become more readily apparent to those with skill in the art with reference to the following detailed description of the preferred embodiments and the accompanying figures and drawings.

### BRIEF DESCRIPTION OF THE DRAWINGS

Without limiting the scope of the dental orthotic strut as disclosed herein and referring now to the drawings and figures:

FIG. 1 is a front perspective view of an embodiment of an anti-obstructive airway dental orthotic device producing increased intraoral volume;

FIG. 2A is a side perspective view of the dental orthotic device of FIG. 1;

FIG. 2B is another side perspective view of the dental orthotic device of FIG. 1;

FIG. 3A is a side perspective view of the dental orthotic device of FIGS. 1 and 2 shown with the strut removed;

FIG. 3B is a bottom (cephalic facing) view of the maxillary retainer of the orthotic device of FIG. 3A;

FIG. 4 is a bottom (cephalic facing) view of an embodiment of a maxillary retainer of the dental orthotic device of FIGS. 1-3;

FIG. 5 is a top (caudal facing) view of a mandibular retainer of the anti-obstructive airway dental orthotic device of FIGS. 1-3;

FIG. 6 is a bottom (cephalic facing) view of another embodiment of a maxillary retainer of an anti-obstructive airway dental orthotic device; and

FIG. 7 is a top (caudal facing) view of another embodiment of a mandibular retainer of an anti-obstructive airway dental orthotic device;

FIG. 8 is a top plan view of a maxillary retainer overlying a mandibular retainer of the anti-obstructive airway dental orthotic device of FIGS. 1-3;

FIG. 9 is a detail view showing the interaction between a first and second maxilla to strut pivot points external diameters, a strut to maxilla pivot point internal diameter, a strut to mandible pivot point internal diameter, and a mandible to strut pivot point external diameter showing the diameter relationships among these structures; and

FIG. 10 is an embodiment of an anterior retainer according to the present invention.

These drawings are provided to assist in the understanding of the exemplary embodiments of the anti-obstructive airway dental orthotic producing increased intraoral volume as described in more detail below and should not be construed as unduly limiting the dental orthotic. In particular, the relative spacing, positioning, sizing and dimensions of the various elements illustrated in the drawings are not drawn to scale and may have been exaggerated, reduced, or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

The disclosed anti-obstructive airway dental orthotic producing increased intraoral volume enables a significant advance in the state of the art. The preferred embodiments of the dental orthotic accomplish this by new and novel arrangements of elements and methods that are configured in unique and novel ways, and which demonstrate previously unavailable but preferred and desirable capabilities. The description set forth below in connection with the drawings is intended merely as a description of the presently preferred embodiments of the dental orthotic and is not intended to represent the only form in which the dental orthotic may be constructed or utilized. The description sets forth the designs, functions, means, and methods of implementing the dental orthotic in connection with the illustrated embodiments. It is to be understood, however, that the same or equivalent functions and features may be accomplished by different embodiments that are also intended to be encompassed within the spirit and scope of the claimed dental orthotic.

As described throughout this specification the terms anterior and posterior shall describe relative positions to each other and shall mean as follows: Anterior shall mean more distant from a coronal, or frontal plane, relative to the term posterior, which shall mean closer to a coronal, or frontal plane. The terms cephalic (towards the head) and caudal (towards the feet) shall have the usual medical meanings. The terms lingual (toward the tongue), buccal (towards the cheek), occlusal (towards the opposing tooth or teeth), and cervical shall have the usual meaning as applied in dentistry. Additionally, the terms “clinician” or “practitioner” shall mean any person practicing the invention, which may be, by way of example and not limitation, any one of a wide variety of health care practitioners. The term “patient” or “user” shall mean any human subject employing the use of the device for any purpose whatsoever. One skilled in the art will realize the essentially variable nature of human physiology, and that therefore the use of the terms, “about,” “approximate,” or “approximately,” in the specification and claims must necessarily be subject to natural variation. However, it has been experienced that a range of plus or minus 20% will include the great majority of human variants, although this is not absolutely so. Accordingly, such a variation should be applied to all figures and measurements given.

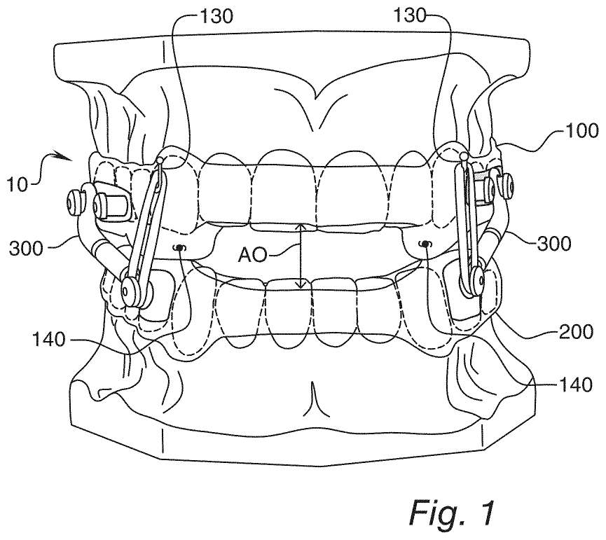

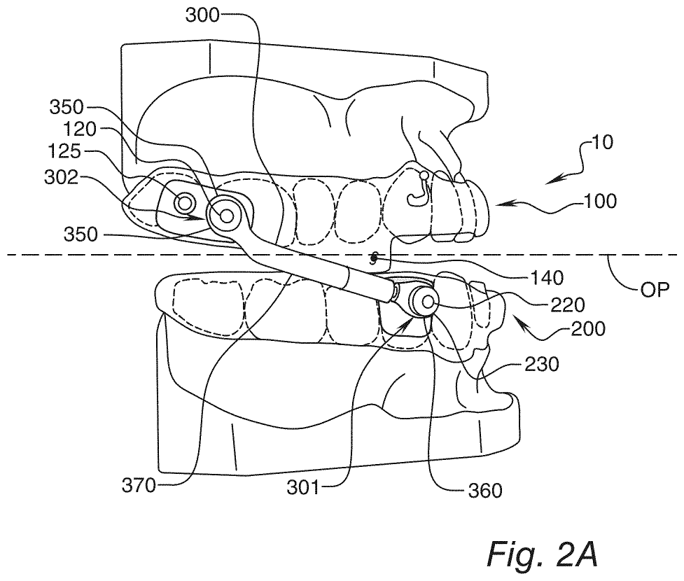

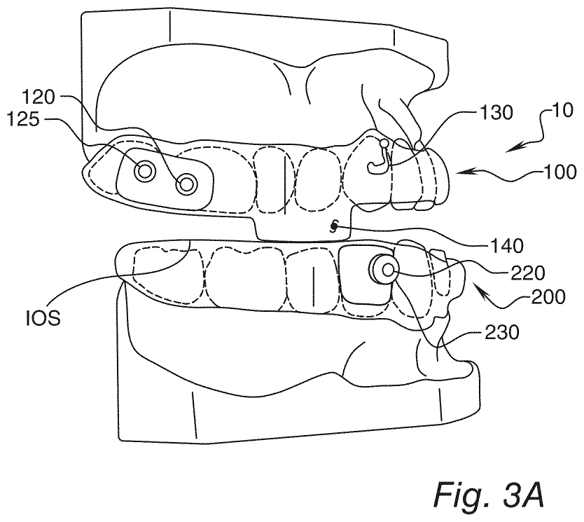

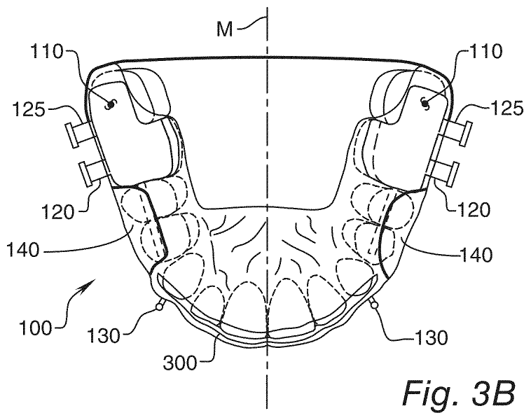

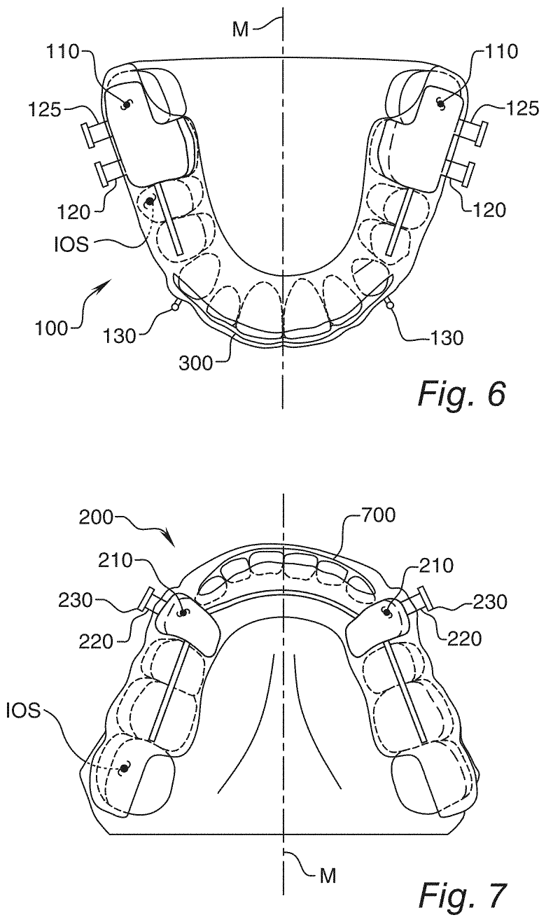

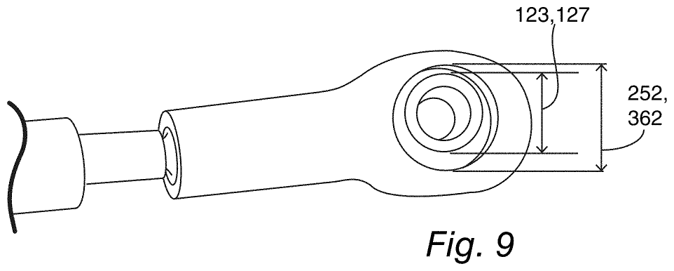

What is claimed then, and is illustrated well in FIGS. 1-10, is an anti-obstructive airway dental orthotic (10) providing increased intraoral volume that includes a rigid strut (300) having a strut body (370) partially containing a strut piston (380). The piston (380) has free and unbiased axial movement between only a strut (300) fixed minimum length and only a strut (300) fixed maximum length, seen well in FIGS. 1-2B. This is in itself a major departure from the prior art, where the struts of devices intended to advance the lower jaw relative to the upper are nearly all either a) adjustable to a plurality of intermediate lengths between a minimum and a maximum length (the present devices having no intermediate adjustable lengths), or b) biased, such as by internal spring means, to exert expansile or contractive pressure. The strut (300) may have an anterior end (301) having a rotatable strut to mandible pivot point (360) formed as an at least partially closed loop having an internal diameter, as well as a posterior end (302) having a rotatable strut to maxilla pivot point (350) formed as an at least partially closed loop having an internal diameter, both seen well in FIG. 9. For this specification, the term “partially closed” means a loop that has a break in the loop circumference. In a preferred embodiment, this break represents less than 50% of the total circumference of the loop. In another preferred embodiment, the “at least partially closed” loop is fully closed in its circumference, as seen well in FIG. 9.

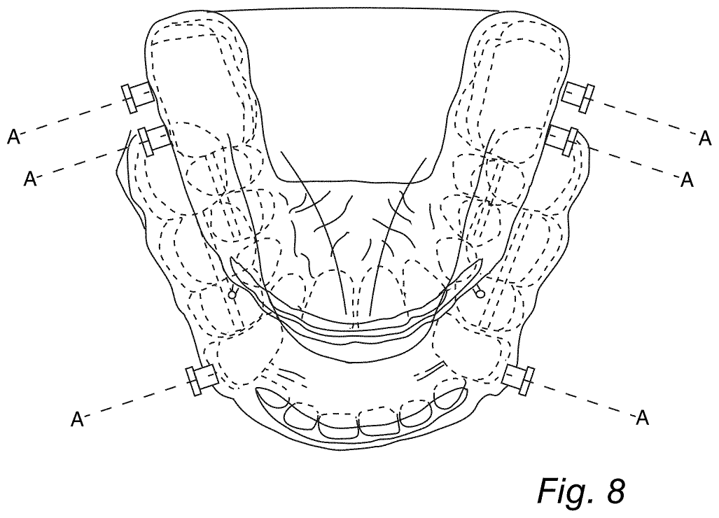

The orthotic (10) includes a maxillary retainer (100), seen well in FIGS. 1-4, 6 and 8, for cooperating with and reversibly attaching to a plurality of maxillary teeth of a user, wherein the maxillary retainer (100) includes four maxillary to strut pivot points (120, 125) formed as posts. These four posts include a left-side first maxilla to strut pivot point (120), a right-side first maxilla to strut pivot point (120), a left-side second maxilla to strut pivot point (125) and a right-side second maxilla to strut pivot point (125), also seen well in FIGS. 1-4, 6, and 8. Both pivot points on each respective side (120, 125) of the mandibular retainer (100) have parallel axes of rotation, as can be seen in FIG. 8.

The orthotic also has a mandibular retainer (200) for cooperating with and reversibly attaching to a plurality of mandibular teeth of the user, seen well in FIGS. 1-3A, 5, 7, and 8. The mandibular retainer (200) includes two mandibular to strut pivot points (220) formed as posts comprising a left-side mandible to strut pivot point (220) and a right-side mandible to strut pivot point (220), also seen well in FIGS. 1-3B, 5, 7, and 8. As can be seen well in FIGS. 1, 4, 6, and 8, the maxillary to strut pivot points (120, 125) are all posterior, relative to a frontal plane, to the mandible to strut pivot points (220). As seen well in FIG. 8, the left side mandibular pivot point (220) has a parallel axis of rotation to both left side maxillary pivot points (120, 125) and the right side mandibular pivot point (220) has a parallel axis of rotation to both right side maxillary pivot points (120, 125), This, at least in some embodiments, is an important design factor, as the parallel relationship between both end points of the strut (300) connections is important to the smooth and non-binding operation of the strut (300).

The strut to maxilla pivot point (350) rotatably cooperates with at least one of the first maxilla to strut pivot points (120) and the second maxilla to strut pivot points (125) to rotatably and reversibly connect the maxillary retainer (100) and the strut (300) at a rotating mechanical joint to reversibly attach and detach the strut (300) from the retainer (100). Similarly, the strut to mandible pivot point (360) rotatably cooperates with at least one of the mandible to strut pivot points (220) to rotatably and reversibly connect the mandibular retainer (200) and the strut (300) at a rotating mechanical joint to reversibly attach and detach the strut (300) from the retainer (200). Both features are seen well in FIGS. 1-2B.

Again, with reference to FIG. 8, and for the same reasons as elucidated above, that is, smooth and non-binding motion between the retainers (100, 200) and the strut (300), all left-side pivot points (120, 125, 220) have singular, fixed axes of rotation, and all such left-side pivot points (120, 125, 220) axes of rotation lie in parallel planes, and all right-side pivot points (120, 125, 220) have singular, fixed axes of rotation, and all such right-side pivot points (120, 125, 220) axes of rotation lie in parallel planes.

The net effect of the arrangements above is that, when a jaw of a user is closing or fully closed, the mandibular retainer (100) and the maxillary retainer (200) are approximated relative to an imaginary occlusal plane (OP)(being defined as an imaginary plane passing between and parallel to the occlusal surfaces of the maxillary and mandibular teeth, as seen well in FIGS. 2A-2B), and the strut (300) creates an expansile force between the maxillary (100) and mandibular (200) retainers.

In some embodiments, the posts the posts are hollow, internally threaded posts; however, one skilled in the art would be able to envision solid, or externally threaded posts, or even other means for linking the strut (300) to the retainers (200).

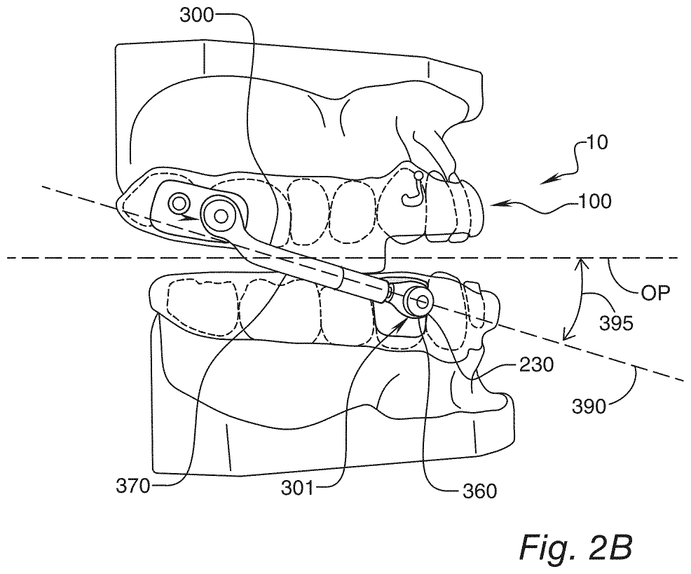

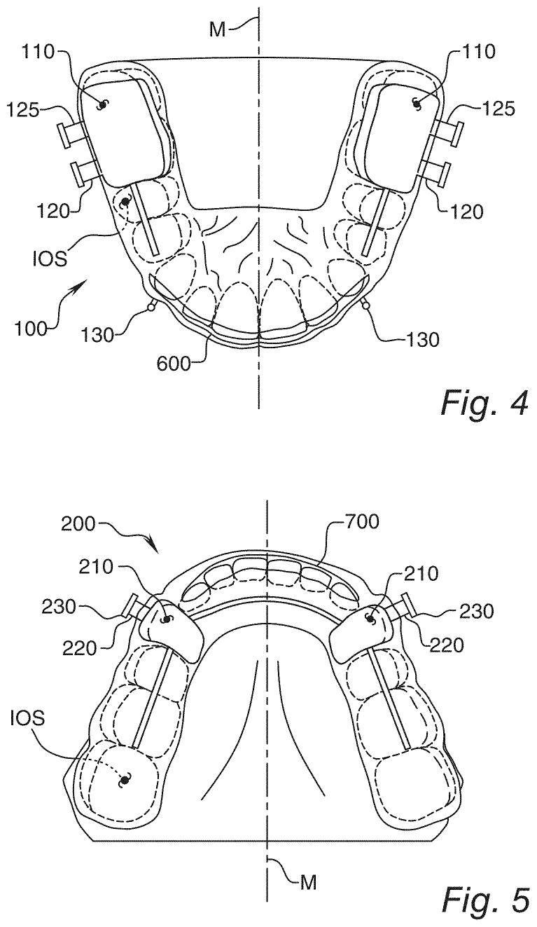

As seen well in FIGS. 1-7, for purposes of increased strength. each of the left-side and right-side pivot points (120, 125, 220) maybe attached to a reinforcement (110, 210) embedded within the respective maxillary retainer (100) and mandibular retainer (200). These reinforcements may be configured as substantially L-shaped reinforcements (110, 210), seen well in FIGS. 3B and 6, each having an occlusal limb and a buccal limb to which the pivot points (120, 125, 220) attach. In some embodiments, the posts (120, 125, 220) are fixed to the reinforcements. In one preferred embodiment, the posts (120, 125, 220) are laser-welded to the reinforcements (110, 210) This is neither an intuitive point nor one that is susceptible to routine experimentation. A standard practice in the dental arts is for metallic components to be soldered to one another for ease of assembly. However, soldering introduced foreign substances such as solder and flux into the devices, and these substance are often highly irritating and even allergenic. In many common embodiments of the present invention(s), the reinforcements and post are formed of very non-allergenic materials, such as, by way of example only and not limitation, 316L surgical stainless steel. Laser welding can fuse the 316L posts to the 316L reinforcements without the addition of exogenous substances, thereby making the fabrication as free of potential irritants or allergens as possible.

In one particular set of embodiments, each of the left-side maxillary pivot points (120, 125) and each of the right-side maxillary pivot points (120, 125) may have an anterior-posterior separation, from each other, measured along the imaginary occlusal plane (OP), of about 4 millimeters. Again, this limitation is neither intuitive nor susceptible to routine experimentation. It has been found that this distance may be about the minimum required for free movement of the strut (300) and the maxilla to strut pivot points (120, 125), and may also be ideal for a suitable advancement of the jaw when the strut (300) is moved from the second maxilla to strut pivot point (125) to the first maxilla to strut pivot point (120) as part of the advancement process.

In some embodiments, at least one of the maxillary retainer (100) and the mandibular retainer (200) has an interior buccal surface, an interior lingual surface, and an interior occlusal surface (IOS), such that the interior buccal and internal lingual surfaces are molded, in the area of a plurality of teeth, to be separated at a retainer edge closest to the imaginary occlusal plane (OP) by a distance equal to a medial to lateral cervical width of each of the plurality of teeth to which the retainer (100, 200) is approximated. The IOS may be relatively flat, touching only the occlusal peaks of the underlying teeth, or may be molded to conform to the full occlusal contours of the underlying teeth. This confers a significant benefit in these embodiments. As one skilled in the art will know, the cervical width of a tooth is generally slightly less than the width of the same tooth at the maximum width of the enameled area. Thus, in these embodiments, the slightly narrow, relative to the enameled surface diameter of the tooth, gap between the buccal and lingual internal surfaces of the orthotic (10), at the retainer edge closest to the imaginary occlusal plane (OP), that is, the point of the orthotic (10) that must be pushed over and onto the teeth, results, due to a slight resilience of the orthotic (10), in the orthotic “snapping” onto the tooth for increased holding power and better fit.

In other embodiments, but not necessarily all, the maxillary retainer (100) has a spacing section (140) comprising an area of increased thickness such that in use, a minimum vertical separation is produced between a cusp of the user's maxillary premolar and a cusp of the user's opposing mandibular premolar of approximately between 2 and 5 millimeters. Once again, this feature is neither intuitive nor susceptible to routine experimentation. In terms of airway patency, it can be said that “volume is everything.” Anything that increases the free volume of the upper airway is helpful to the sleep apnea, or otherwise partially obstructed, patient. A major determinant of this free volume, and even perhaps a major impediment to increasing the same, is the patient's tongue. In many, but not all further embodiments, the spacing section (140) is approximately 12-18 millimeters in anterior-posterior length and is approximately centered over an area inferior to a first and second premolar tooth, may have a lateral margin coextensive with a lateral margin of the maxillary retainer, and may also have a medial to lateral width of approximately 5-7 millimeters. Each of these limitations confers, both alone and acting together, important advantages.

A spacing section (140) of approximately 12-18 millimeters in anterior-posterior length creates a lateral “tongue space” both in front of and behind the spacing section, as can be envisioned from FIGS. 3A-3B. This space, held open by the spacing section (140), allows the tongue to protrude slightly into said space, between the teeth, thereby moving more of the tongue out of the airway.

Centering the spacing section immediately inferior to the first and second premolars has been found to produce optimization of the extra space produced both in front of and behind the spacing section (140). Having the spacing section (140) coextensively share a lateral border with the lateral margin of the maxillary retainer (100), that is, both the spacing section (140) and the maxillary retainer (100) extend the same distance laterally, generally but not necessarily in at least a mostly smooth and unbroken manner, further increases the intraoral free volume by moving as much of the spacing section (140) laterally as feasible. Lastly, having the spacing section (140) be only 5-7 millimeters wide, when one skilled in the art will know that the average occlusal width of the pre-molar teeth is approximately 10 millimeters, means that the spacing section (140) has only approximately 50-70% of the width of the underlying teeth. Thus, even more volume is created along the medial border of the spacing section (140), seen well in FIG. 3B, which again promotes lateral movement of the tongue and an increase in the free intraoral volume.

The strut (300) may have a fixed minimum length of between 25 and 29 millimeters, measured between a center point of the strut to maxilla pivot point (350) and the strut to mandible pivot point (360). When the strut (300) is attached to both the maxillary retainer (100) and the mandibular retainer (200) and the retainers (100, 200) are approximated in use in a human patient, a strut angulation (395) of the strut (300) of approximately 15-22 degrees between a lengthwise strut axis (390) and the imaginary occlusal plane (OP) is produced. This is seen well in FIG. 2B and has been found to be ideal in effecting both lower jaw advancement and patient comfort and utility. Once again, this seemingly arbitrary figure is not arbitrary at all, as it has been found that lesser angulations can be less effective in securing advancement, while greater angulations make it hard for the patient to close the jaw.

In practice, and using a non-adjustable length strut (300) and only two discrete maxilla to strut pivot points (120, 125), when the strut to maxilla pivot point (350) is attached to one of the second maxilla to strut pivot point (125) and (subsequently to) the first maxilla to strut pivot point (120), and the strut to mandible pivot (360) is attached to the mandible to strut pivot point (220) on both the left and right sides of the respective retainers (100, 200), an anterior mandible advancement beyond a point of habitual occlusion of approximately 3 to 8 millimeters is produced when the retainers (100, 200) are approximated during use. This provides adequate advancement and symptom relief for many patients, and obviates all of the mechanical and fabricating difficulties inherent in the adjustable length struts of the prior art. This, in turn, leads to the startling advance over the prior art that, using a fixed length strut (300) with only two length adjustment points (at the maxilla to strut pivot points, 120, 125) is sufficient to effect considerable improvement in a large number of patients. Thus, the need for an adjustable length strut is largely eliminated.

Jaw displacement indicia, as one skilled in the art would know, may be, if desired, marked on the retainers (100, 200) such that any displacement of the retainers (100, 200) from a point of habitual occlusion can be quantified. In some embodiments, these take the form of vertical lines, one on the maxillary retainer (100) and one on the mandibular retainer (200), scribed or otherwise placed directly above and below each other when the retainers (100,200) are in place and the jaw is at the point of habitual occlusion. One skilled in the art will, of course, easily envision other indicia that may aid in quantifying displacement.

Again, following the dicta of “volume is everything,” the orthotic (10) may be fabricated to have an anterior maxillary fenestration (600) and an anterior mandibular fenestration (700), allowing the exposure of the occlusal surfaces of at least eight teeth, comprising both upper and lower central incisors and both upper and lower lateral incisors. Even the removal of the small amount of orthotic (10) material that covers the occlusal surface of these teeth, and therefore ever-so-slightly may obstruct the airway, has shown to be useful, without compromising the secure attachment of the orthotic (10) to the teeth.

Another important design feature in some, but not all embodiments, involves the attachments formed between the retainers (100, 200) and the strut (300). In its simplest expression, this attachment may be in the form of an at least partially closed loop (350, 360) on the strut (300) fitted over a corresponding post (120, 125, 220) on the retainer (100, 200). It has been found that making this fit mechanically closely-fitted decreases the function of the device. In the event of even slight misalignments, a “tight fit” can cause the rotational joint to bind, which causes patient discomfort and even threatens the integrity of the orthotic (10). Therefore, in many, but not all, embodiments, the posts (120, 125, 220) on the maxillary and mandibular retainers (100, 200) each may have an external diameter (122, 127, 222) that is approximately 0.8-1.2 millimeters smaller than an internal diameter (352, 362) of each of the at least partially closed loops on the strut (300). This allows for a “sloppy” yet strong fit with about 0.5 millimeters of free space between each point on the sides of the posts (120, 125, 220) and their corresponding at least partially closed loops (352, 362).

Of course, especially with such a “loose-fit” situation, the at least partially closed loops (352, 362) must be retained onto the posts (120, 125, 220) lest they otherwise fall off their respective attachments. In many embodiments, but again not all, this is accomplished by a closure, by way of example only and not limitation, formed as a threaded screw, having a head with a diameter greater than the internal diameter of the at least partially closed loops. This screw can be fabricated so as to be slightly longer than a medial to lateral thickness of the underlying at least partially closed loop (352, 362), so again, some “slop” in an axial direction to the post (1120, 125, 220) is produced, even when the screw may be fully tightened, adding to the “slop” in the lateral dimension already produced, again decreasing the chance for binding of the joint.

The orthotic described above in the specification and drawings, and in the claims below, is useful in a variety of methods used to increase airway patency. One such method is for the reduction of Obstructive Sleep Apnea (OSA), comprising the steps of:

First, performing a baseline evaluation of a patient's medical, dental and sleep history, and then fabricating an anti-obstructive airway dental orthotic (10). Such an orthotic can include a rigid strut (300) of predetermined fixed minimum length that is freely extendable between a predetermined minimum length and a predetermined maximum length. Such a strut might have an anterior end (301) having a rotatable strut to mandible pivot point (360), and a posterior end (302) having a rotatable strut to maxilla pivot point (350).

A maxillary retainer (100) would be included in this orthotic (10) for cooperating with and reversibly attaching to a plurality of maxillary teeth of a user, including an area of increased thickness such that in use, a minimum vertical separation of approximately 2-5 millimeters is produced between a cusp of the user's maxillary premolars and a cusp of the user's opposing mandibular premolars. This area of increased thickness may be identical to, similar to, or completely different form the “spacing section (140)” described above.

The maxillary retainer (100) can include a first maxilla to strut pivot point (120) and a second maxilla to strut pivot point (125), each having a central axis of rotation about the respective maxilla to strut pivot point (120, 125), the pivot points (120, 125) having an anterior-posterior separation of about 4 millimeters measured center-to-center along an imaginary line drawn parallel to an occlusal surface of the maxillary retainer (100).

The orthotic (10) can also include a mandibular retainer (200) for cooperating with and reversibly attaching to a plurality of mandibular teeth of the user, where, analogously to the maxillary retainer (100), the mandibular retainer (200) may include a mandible to strut pivot point (220) having a central axis of rotation about the respective mandible to strut pivot point (220). In such a construction, the central axis of rotation of the at least a first maxilla to strut pivot point (120), the at least a second maxilla to strut pivot point (125), and the mandible to strut pivot point (220) may all lie in parallel planes, as seen well in FIG. 8.

Attaching the various components together initially, the strut to maxilla pivot point (350) may rotatably cooperate with at the second maxilla to strut pivot point (125) to rotatably and reversibly connect the maxillary retainer (100) and the strut (300), and the strut to mandible pivot point (360) may rotatably cooperate with the mandible to strut pivot point (220) to rotatably and reversibly connect the mandibular retainer (200) and the strut (300).

The net result is such that in use, as the jaw is closing or closed, with the maxillary retainer (100) cooperating with and reversibly attached to the plurality of maxillary teeth of the user and the mandibular retainer (200) cooperating with and reversibly attached to the plurality of mandibular teeth of the user, an expansive force is produced between the maxilla and the mandible is produced. When the strut (300) is attached to the second (i.e., most posterior) maxilla to strut pivot point (125), an anterior mandible advancement of approximately 2-4 millimeters from a point of habitual occlusion is produced. It is to be noted that the maxillary to strut pivot points (120, 125) are all posterior, relative to a frontal plane, to the mandible to strut pivot points (220).

Following steps could include reversibly applying the orthotic (10) to the maxillary and mandibular teeth of the patient; verifying the fit and comfort of the orthotic (10) to the patient; waiting a predetermined period of time and making periodic assessments of a constellation of the patient's subjective symptoms and evaluating and adjusting the separation between the retainers (100, 200) caused by the area of increased thickness (140) described above.

In a progression of treatment, moving the strut (300) from the second maxilla to strut pivot point (125) to the first maxilla to strut pivot point (120), a further expansive force is produced between the maxilla and the mandible, resulting in a total anterior mandible advancement of approximately 4-8 millimeters from the point of habitual occlusion. Treatment could then continue with waiting a predetermined period of time and making periodic assessments of a constellation of the patient's subjective symptoms, and then repeating such of the steps given above as required for satisfactory relief of symptoms.

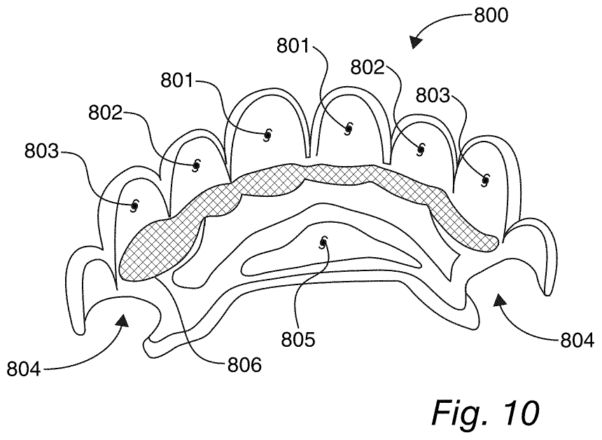

Clinicians employing the orthotic (10) occasionally are confronted with patients describing aching, and even spasm, of the jaw muscles, especially when first employing the orthotic (10). In some cases, it has been found helpful to provide an anterior retainer (800), seen well in FIG. 10, covering an anterior aspect of the maxillary central incisors (801), lateral incisors (802) and canines (803). This retainer is often fabricated of a relatively hard (and therefore slippery), and oftentimes clear, plastic. When the patient's jaw is closed, the anterior retainer (800), contacting the palate (805) and contacting the occlusal surfaces (808) of the mandibular central incisors, lateral incisors, and canines, often provides relief from such complaints, although the exact mechanism of this relief is not yet fully known. The anterior retainer (800) is anchored to the maxillary first premolar (804) but does not contact the occlusal surface of the first maxillary premolar, first mandibular premolar, or any tooth posterior to the pre-molars, and in fact, any such contact has been found to decrease the effectiveness of the anterior retainer (800) The anterior retainer (800) is to be worn by the patient at all times other than when sleeping.

In a further treatment step, elastic devices (400), often such as rubber bands, may be provided to be worn between a left maxillary closure attachment (130) and a left mandible closure attachment (230), and between a right maxillary closure attachment (130) and a right mandible closure attachment (230) while the orthotic (10) is in use, as seen well in FIG. 1.

Patients with even minor levels of airway obstructive are well-known to suffer increased perioperative morbidity and mortality, and these dangers are increasing all the time due to the increased utilization of outpatient, or “same day” surgery, where post-operative observation and supervision of the patient is attenuated.

Accordingly, the orthotic (10) described herein has been found useful in a pre-operative protocol that employs a method of increasing airway patency prior to surgery, and thereby to decrease surgically related complications.

This method can, but is not limited to, include the steps of first, scanning the teeth and oral cavity of a patient with a digital scanner to map intraoral topography and relationships, and then fabricating an orthotic (100), customized to a patient using laser printing, that advances a lower jaw relative to an upper jaw.

Next, over a predetermined time period prior to surgery, the following steps may occur: Fitting the orthotic to a patient approximately ten to fourteen days prior to surgery; and instructing the patient to wear the orthotic (10) at all times when sleeping. Also, a clinician may instruct the patient to wear an anterior retainer (800), covering an anterior aspect of the maxillary central incisors (801), lateral incisors (802) and canines (803), and which, when the patient's jaw is closed, contacts the occlusal surfaces (808) of the mandibular central incisors, lateral incisors, and canines, anchored to the maxillary first premolar. Such an anterior retainer (800) may be worn by the patient at all times other than when sleeping.

The method has been found to have increased efficacy by instructing the patient to use elastic devices (400), often rubber bands, worn between a left maxillary closure attachment (130) and a left mandible closure attachment (230), and between a right maxillary closure attachment (130) and a right mandible closure attachment (230) while the orthotic (10) is in use, as seen in FIG. 1.

After 10-14 days of use, or after some other period of time as determined by the clinician, the clinician may reset the strut (300) to a first and more anterior maxillary pivot point (120), thereby increasing the jaw advancement. Later, surgical interventions as required may be performed, removing the orthotic (10) during said surgery.

Immediately post-operatively, the orthotic (10) is reinserted and normal post-operative parameters may be monitored. Final steps include instructing the patient, after discharge from a surgical area, to resume wearing of the orthotic (10) while sleeping and to wear the anterior retainer (800) at all times when not sleeping.

The orthotic (10) is inspected for fit, adjustment, and integrity at all postoperative patient visits, and at approximately 30 days postoperatively, or at some other time as may be determined by the clinician, use of the orthotic (10) is discontinued, and/or the patient is referred to an orthotic-trained medical practitioner for treatment of obstructive sleep apnea.

Thus, the process could involve waiting a predetermined period of time and again making periodic assessments of the constellation of the patient's subjective and objective symptoms, and then some of the steps above may be repeated as required for a satisfactory relief of symptoms.

Numerous alterations, modifications, and variations of the preferred embodiments disclosed herein will be apparent to those skilled in the art and they are all anticipated and contemplated to be within the spirit and scope of the disclosed anti-obstructive airway dental orthotic producing increased intraoral volume (10). For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the dental orthotic (10) are described herein, it is to be understood that the practice of such additional modifications and variations and the equivalents thereof, are within the spirit and scope of the dental orthotic (10) as disclosed herein. The corresponding structures, materials, acts, and equivalents of all means or step plus function elements in the claims below are intended to include any structure, material, or acts for performing the functions in combination with other claimed elements as specifically claimed.

I claim:

1. An anti-obstructive airway dental orthotic (10), comprising:
a rigid strut (300) having a strut body (370) partially containing a strut piston (380) wherein the piston (380) has free and unbiased axial movement between only a strut (300) fixed minimum length and only a strut (300) fixed maximum length, an anterior end (301) having a rotatable strut to mandible pivot (360) formed as an at least partially closed loop having an internal diameter, and a posterior end (302) having a rotatable strut to maxilla pivot (350) formed as an at least partially closed loop having an internal diameter,
a maxillary retainer (100) for cooperating with and reversibly attaching to a plurality of maxillary teeth of a user, wherein the maxillary retainer (100) includes four maxilla to strut pivot points (120, 125) formed as posts comprising a left-side first maxilla to strut pivot point (120), a right-side first maxilla to strut pivot point (120), a left-side second maxilla to strut pivot point (125) and a right-side second maxilla to strut pivot point (125), both pivot points on each respective side (120, 125) of the mandibular retainer (100) having parallel axes of rotation,
a mandibular retainer (200) for cooperating with and reversibly attaching to a plurality of mandibular teeth of the user, wherein the mandibular retainer (200) includes two mandible to strut pivot points (220) formed as posts comprising a left-side mandible to strut pivot point (220) and a right-side mandible to strut pivot point (220),
the maxilla to strut pivot points (120, 125) being all posterior, relative to a frontal plane, to the mandible to strut pivot points (220),
the left side mandibular pivot point (220) having a parallel axis of rotation to both left side maxillary pivot points (120, 125) and the right side mandibular pivot point (220) having a parallel axis of rotation to both right side maxillary pivot points (120, 125),
the strut to maxilla pivot point (350) rotatably cooperates with at least one of the first maxilla to strut pivot points (120) and the second maxilla to strut pivot points (125) to rotatably and reversibly connect the maxillary retainer (100) and the strut (300) at a rotating mechanical joint to reversibly attach and detach the strut (300) from the retainer (100),
the strut to mandible pivot point (360) rotatably cooperates with at least one of the mandible to strut pivot points (220) to rotatably and reversibly connect the mandibular retainer (200) and the strut (300) at a rotating mechanical joint to reversibly attach and detach the strut (300) from the retainer (200),
all left-side pivot points (120, 125, 220) have singular, fixed axes of rotation, and all such left-side pivot points (120, 125, 220) axes of rotation lie in parallel planes, and all right-side pivot points (120, 125, 220) have singular, fixed axes of rotation, and all such right-side pivot points (120, 125, 220) axes of rotation lie in parallel planes, such that,
when a jaw of a user is closing or fully closed, the mandibular retainer (100) and the maxillary retainer (200) are approximated relative to an imaginary occlusal plane (OP), and the strut (300) creates an expansile force between the maxillary (100) and mandibular (200) retainers.

2. The device according to claim 1, wherein the posts are hollow, internally threaded posts.

3. The device according to claim 1, wherein each of the left-side and right-side pivot points (120, 125, 220) are attached to a reinforcement (110, 210) embedded within the respective maxillary retainer (100) and mandibular retainer (200), said reinforcements configured as substantially L-shaped reinforcements (110, 210) each having an occlusal limb, and a buccal limb to which the pivot points (120, 125, 220) are laser welded.

4. The device according to claim 1, wherein the left-side maxillary pivot points (120, 125), have an anterior-posterior separation, measured along the imaginary occlusal plane (OP) of about 4 millimeters and the right-side maxillary pivot points (120, 125) each have an anterior-posterior separation, measured along the imaginary occlusal plane (OP) of about 4 millimeters.

5. The device according to claim 1, wherein at least one of the maxillary retainer (100) and the mandibular retainer (200) has an interior buccal surface, an interior lingual surface, and an interior occlusal surface (IOS), wherein the interior buccal and internal lingual surfaces are molded, in the area of a plurality of teeth, to be separated at a retainer edge closest to the imaginary occlusal plane (OP) by a distance equal to a medial to lateral cervical width of each of the plurality of teeth to which the retainer (100, 200) is approximated.

6. The device according to claim 1, wherein the maxillary retainer (100) has a spacing section (140) comprising an area of increased thickness such that in use, a minimum vertical separation is produced between a cusp of the user's maxillary premolar and a cusp of the user's opposing mandibular premolar of approximately 2-5 millimeters.

7. The device according to claim 6, wherein the spacing section (140) is approximately 12-18 millimeters in anterior-posterior length and is approximately centered over an area inferior to a first and second premolar tooth.

8. The device according to claim 6, wherein the spacing section (140) has a lateral margin coextensive with a lateral margin of the maxillary retainer, and a medial to lateral width of approximately 5-7 millimeters.

9. The device according to claim 1, wherein the strut (300) has a fixed minimum length of between 25 and 29 millimeters, measured between a center point of the strut to maxilla pivot point (350) and a center point of the strut to mandible pivot point (360), and when the strut (300) is attached to both the maxillary retainer (100) and the mandibular retainer (200) and the retainers (100, 200) are approximated in use in a human patient, a strut angulation (395) of the strut (300) of approximately 15-22 degrees between a lengthwise strut axis (390) and the imaginary occlusal plane (OP) is produced.

10. The device according to claim 1, wherein when the strut to maxilla pivot (350) is attached to one of the first maxilla to strut pivot point (120) and the second maxilla to strut pivot point (125), and the strut to mandible pivot (360) is attached to the mandible to strut pivot point (220) on both the left and right sides of the respective retainers (100, 200), an anterior mandible advancement beyond a point of habitual occlusion of approximately 3 to 8 millimeters is produced when the retainers (100, 200) are approximated during use.

11. The device according to claim 1, wherein jaw displacement indicia (500) are marked on the retainers such that any displacement of the retainers from a point of habitual occlusion can be quantified.

12. The device according to claim 1, wherein the orthotic (10) has an anterior maxillary fenestration (600) and an anterior mandibular fenestration (700), allowing the exposure of the occlusal surfaces of at least eight teeth, comprising both upper and lower central incisors and both upper and lower lateral incisors.

13. The device according to claim 1, wherein the posts (120, 125, 220) on the maxillary and mandibular retainers (100, 200) each have an external diameter (122, 127, 222) that is approximately 0.8-1.2 millimeters smaller than an internal diameter (352, 362) of each of the at least partially closed loops on the strut (300).

14. The device according to claim 13, wherein each the at least partially closed loops of the strut (300) are affixed over the posts by a closure having a head with a diameter greater than the internal diameter of the at least partially closed loops.

15. An anti-obstructive airway dental orthotic (10), comprising:
a rigid strut (300) having a strut body (370) partially containing a strut piston (380) wherein the piston (380) has free and unbiased axial movement between only a strut (300) fixed minimum length and only a strut (300) fixed minimum length of between 25 and 29 millimeters, measured between a center point of a strut to maxilla pivot point (350) and a strut to mandible pivot point (360), and when the strut (300) is attached to both the maxillary retainer (100) and the mandibular retainer (200) and the retainers (100, 200) are approximated in use in a human patient, a strut angulation (395) of approximately 15-22 degrees between a lengthwise strut axis (390) and an imaginary occlusal plane (OP) is produced,
an anterior end (301) having the rotatable strut to mandible pivot point (360) formed as an at least partially closed loop having an internal diameter, and a posterior end (302) having the rotatable strut to maxilla mechanical pivot point (350) formed as an at least partially closed loop having an internal diameter,
a maxillary retainer (100) for cooperating with and reversibly attaching to a plurality of maxillary teeth of a user, wherein the maxillary retainer (100) includes four maxilla to strut pivot points (120, 125) formed as threaded posts comprising a left-side first maxilla to strut pivot point (120), a right-side first maxilla to strut pivot point (120), a left-side second maxilla to strut pivot point (125) and a right-side second maxilla to strut pivot point (125), both pivot points on each respective side (120, 125) of the mandibular retainer (100) having parallel axes of rotation and having an anterior-posterior separation, measured along the imaginary occlusal plane (OP) of about 4 millimeters,
the maxillary retainer (100) having a spacing section (140) comprising an area of increased thickness that is approximately 12-18 millimeters in anterior-posterior length, is approximately centered over an area inferior to a first and second premolar tooth, having a lateral margin coextensive with a lateral margin of the maxillary retainer, and a medial to lateral width of approximately 5-7 millimeters, such that in use, a minimum vertical separation is produced between a cusp of the user's maxillary premolar and a cusp of the user's opposing mandibular premolar of approximately between 2 and 5 millimeters,
a mandibular retainer (200) for cooperating with and reversibly attaching to a plurality of mandibular teeth of the user, wherein the mandibular retainer (200) includes two mandible to strut pivot points (220) formed as threaded posts comprising a left-side mandible to strut pivot point (220) and a right-side mandible to strut pivot point (220),
the maxilla to strut pivot points (120, 125) being all posterior, relative to a frontal plane, to the mandible to strut pivot points (220),
the threaded posts on the maxillary and mandibular retainers (100, 200) each have an external diameter (122, 127, 222) that is approximately 0.8-1.2 millimeters smaller than an internal diameter (352, 362) of the at least partially closed loops on the strut (300),
the left side mandibular pivot point (220) having a parallel axis of rotation to both left side maxillary pivot points (120, 125) and the right side mandibular point (220) having a parallel axis of rotation to both right side maxillary pivot points (120, 125),
the posts being attached to a reinforcement (110, 210) embedded within the respective maxillary retainer (100) and mandibular retainer (200), such retainers configured as substantially L-shaped reinforcements (110, 210) each having an occlusal limb, and a buccal limb to which the pivot points (120, 125, 220) are laser welded,
at least one of the maxillary retainer (100) and the mandibular retainer (200) has an interior buccal surface, an interior lingual surface, and an interior occlusal surface (IOS), wherein the interior buccal and internal lingual surfaces are molded, in the area of a plurality of teeth, to be separated at a retainer edge closest to the imaginary occlusal plane (OP) by a distance equal to a medial to lateral cervical width of each of the plurality of teeth,
the strut to maxilla pivot point (350) rotatably cooperates with at least one of the first maxilla to strut pivot points (120) and the second maxilla to strut pivot points (125) to rotatably and reversibly connect the maxillary retainer (100) and the strut (300) at a rotating mechanical joint to reversibly attach and detach the strut (300) from the retainer (100),
the strut to mandible pivot point (360) rotatably cooperates with the mandible to strut pivot point (220) to rotatably and reversibly connect the mandibular retainer (200) and the strut (300) at a rotating mechanical joint to reversibly attach and detach the strut (300) from the retainer (200),
all left-side pivot points (120, 125, 220) having singular, fixed axes of rotation, and all such left-side pivot points (120, 125, 220) axes of rotation lie in parallel planes, and all right-side pivot points (120, 125, 220) have singular, fixed axes of rotation, and all such right-side pivot points (120, 125, 220) axes of rotation lie in parallel planes, and,
when a jaw of a user is closing or fully closed, the mandibular retainer (100) and the maxillary retainer (200) are approximated relative to the imaginary occlusal plane (OP), and the strut (300) approaches its minimum length, creating an expansile force between the maxillary (100) and mandibular (200) retainers,
such that an anterior mandible advancement beyond a point of habitual occlusion of approximately 3 to 8 millimeters is produced when the retainers (100, 200) are approximated during use.

16. An anti-obstructive airway dental orthotic (10), comprising:
a rigid strut (300) having a strut body (370) and a strut piston (380) wherein the piston (380) has free and unbiased axial movement between only a strut (300) fixed minimum length and only a strut (300) fixed maximum length, an anterior end (301) having a rotatable strut to mandible pivot point (360) formed as an at least partially closed loop, and a posterior end (302) having a rotatable strut to maxilla pivot point (350) formed as an at least partially closed loop,
a maxillary retainer (100) for cooperating with and reversibly attaching to a plurality of maxillary teeth of a user, wherein the maxillary retainer (100) includes four maxilla to strut pivot points (120, 125) formed as posts comprising a left-side first maxilla to strut pivot point (120), a right-side first maxilla to strut pivot point (120), a left-side second maxilla to strut pivot point (125) and a right-side second maxilla to strut pivot point (125), both pivot points on each respective side (120, 125) of the mandibular retainer (100) having parallel axes of rotation, and
the maxillary retainer (100) has a spacing section (140) comprising an area of increased thickness that is approximately 12-18 millimeters in anterior-posterior length, is centered over an area inferior to a first and second premolar tooth, has a lateral margin coextensive with a lateral margin of the maxillary retainer, and a medial to lateral width of approximately 5-7 millimeters, such that in use, a minimum vertical separation is produced between a cusp of the user's maxillary premolar and a cusp of the user's opposing mandibular premolar of approximately between 2 and 5 millimeters,
a mandibular retainer (200) for cooperating with and reversibly attaching to a plurality of mandibular teeth of the user, wherein the mandibular retainer (200) includes two mandible to strut pivot points (220) formed as posts comprising a left-side mandible to strut pivot point (220) and a right-side mandible to strut pivot point (220),
the maxillary to strut pivot points (120, 125) being all posterior, relative to a frontal plane, to the mandible to strut pivot points (220), the left side mandibular pivot point (220) having a parallel axis of rotation to both left side maxillary pivot points (120, 125) and the right side mandibular point having a parallel axis of rotation to both right side maxillary pivot points (120, 125),
the strut to maxilla pivot point (350) rotatably cooperates with at least one of the first maxilla to strut pivot points (120) and the second maxilla to strut pivot points (125) to rotatably and reversibly connect the maxillary retainer (100) and the strut (300) at a rotating mechanical joint to reversibly attach and detach the strut (300) from the retainer (100), and
the strut to mandible pivot point (360) rotatably cooperates with the mandible to strut pivot point (220) to rotatably and reversibly connect the mandibular retainer (200) and the strut (300) at a rotating mechanical joint to reversibly attach and detach the strut (300) from the retainer (200),
all left-side pivot points (120, 125, 220) have singular, fixed axes of rotation, and all such left-side pivot points (120, 125, 220) have axes of rotation that lie in parallel planes, and all right-side pivot points (120, 125, 220) have singular, fixed axes of rotation, and all such right-side pivot points (120, 125, 220) have axes of rotation that lie in parallel planes, such that,
when a jaw of a user is closing or fully closed, the mandibular retainer (100) and the maxillary retainer (200) are approximated relative to an imaginary occlusal plane (OP), and the strut (300) creates an expansile force between the maxillary (100) and mandibular (200) retainers.

17. A method for the reduction of Obstructive Sleep Apnea (OSA), comprising the steps of:
a) performing a baseline evaluation of a patient's medical, dental and sleep history;
b) forming an anti-obstructive airway dental orthotic (10), comprising:
1) a rigid strut (300) of fixed maximum length freely extendable between an only a predetermined minimum length and only a predetermined maximum length, an anterior end (301) having a rotatable strut to mandible pivot point (360), and a posterior end (302) having a rotatable strut to maxilla pivot point (350),
2) a maxillary retainer (100) for cooperating with and reversibly attaching to a plurality of maxillary teeth of a user, comprising an area of increased thickness such that in use, a minimum vertical separation of approximately 2-5 millimeters is produced between a cusp of the user's maxillary premolar and a cusp of the user's opposing mandibular premolar, wherein the maxillary retainer (100) includes a first maxilla to strut pivot point (120) and a second maxilla to strut pivot point (125), each having a central axis of rotation about the respective maxilla to strut pivot point (120, 125), having an anterior-posterior separation of about 4 millimeters measured center-to-center along an imaginary line drawn parallel to an occlusal surface of the maxillary retainer (100),
3) a mandibular retainer (200) for cooperating with and reversibly attaching to a plurality of mandibular teeth of the user, wherein the mandibular retainer (200) includes a mandible to strut pivot point (220) having a central axis of rotation about the mandible to strut pivot point (220),
4) wherein the central axis of rotation of the at least a first maxilla to strut pivot point (120), the at least a second maxilla to strut pivot point (125), and the mandible to strut pivot point (220) all lie in parallel planes,
5) the strut to maxilla pivot point (350) rotatably cooperates with the second maxilla to strut pivot point (125) to rotatably and reversibly connect the maxillary retainer (100) and the strut (300), and
6) the strut to mandible pivot point (360) rotatably cooperates with the mandible to strut pivot point (220) to rotatably and reversibly connect the mandibular retainer (200) and the strut (300), such that in use, with the maxillary retainer (100) cooperating with and reversibly attached to the plurality of maxillary teeth of the user and the mandibular retainer (200) cooperating with and reversibly attached to the plurality of mandibular teeth of the user, an expansive force is produced between the maxilla and the mandible is produced, resulting in an anterior mandible advancement of approximately 2-4 millimeters from a point of habitual occlusion is produced, the maxilla to strut pivot points are all posterior, relative to a frontal plane, to the mandible to strut pivot points;
c) reversibly applying the orthotic (10) to the maxillary and mandibular teeth of the patient;
d) verifying the fit and comfort of the orthotic (10) to the patient;
e) waiting a predetermined period of time and making periodic assessments of a constellation of the patient's subjective symptoms;
f) evaluating and adjusting the separation between the retainers (140, 200);
g) moving the strut (300) from the second maxilla to strut pivot point (125) to the first maxilla to strut pivot point (120), such that in use, with the maxillary retainer (100) cooperating with and reversibly attached to the plurality of maxillary teeth of the user and the mandibular retainer (200) cooperating with and reversibly attached to the plurality of mandibular teeth of the user, an expansive force is produced between the maxilla and the mandible is produced, resulting in an anterior mandible advancement of approximately 4-8 millimeters from the point of habitual occlusion being produced;
h) waiting a predetermined period of time and making periodic assessments of a constellation of the patient's subjective symptoms;
i) repeating steps c-i as required for satisfactory relief of symptoms.

18. The method according to claim 17, comprising the further step of providing an anterior retainer (800), covering an anterior aspect of the maxillary central incisors (801), lateral incisors (802) and canines (803), and, when the patient's jaw is closed, contacting the palate (805) and contacting the occlusal surfaces (808) of the mandibular central incisors, lateral incisors, and canines, anchored to the maxillary first premolar (804), but not contacting the occlusal surface of the first maxillary premolar, first mandibular premolar or any tooth posterior to the pre-molars; to be worn by the patient at times other than when sleeping.

19. The method according to claim 17, further comprising the step of providing elastic devices (400) to be worn between a left maxillary closure attachment (130) and a left mandible closure attachment (230), and between a right maxillary closure attachment (130) and a right mandible closure attachment (230) while the orthotic (10) is in use.

20. A method to reduce post-operative sleep apnea risk comprising the steps of:
1. Scanning the teeth and oral cavity of a patient with a digital scanner to map intraoral topography and relationship;
2. fabricating an orthotic (10) customized to a patient using laser printing that advances a lower jaw relative to an upper jaw;
3. fitting the orthotic (10) to a patient;
4. instructing the patient to wear the orthotic (10) at all times when sleeping;
5. instructing the patient to wear an anterior retainer (800), covering an anterior aspect of the maxillary central incisors (801), lateral incisors (802) and canines (803), and, when the patient's jaw is closed, contacting the occlusal surfaces (808) of the mandibular central incisors, lateral incisors, and canines, anchored to the maxillary first premolar, but not contacting the occlusal surface of the first maxillary premolar, first mandibular premolar or any tooth posterior to the pre-molars; is worn by the patient at times other than when sleeping;
6. instructing the patient to use elastic devices (400) worn between a left maxillary closure attachment (130) and a left mandible to strut pivot point (220), and between a right maxillary closure attachment (130) and a right mandible to strut pivot point (220) while the orthotic (10) is in use;
7. after 10-14 days of use, or some other period of time as may be determined by a clinician, resetting the strut (300) to a second and more anterior maxillary pivot point (125);
8. performing surgical interventions as required, removing the orthotic (10) during said surgery;
9. immediately post-operatively, reinserting the orthotic (10) and monitoring normal post-operative parameters;
10. instructing the patient, after discharge from surgical area, to resume wearing of the orthotic (10) while sleeping;
11. instructing the patient to wear the anterior retainer (800) at all times when not sleeping;
12. inspecting and adjusting orthotic (10) at all postoperative patient visits;
13. at 30 days postoperatively, or some other period of time as may be determined by a clinician, discontinuing use of the orthotic (10) and/or referring patient to orthotic trained medical practitioner for treatment of obstructive sleep apnea.
